(19) United States
(12) Patent Application Publication
(10) Pub. No.: US 2009/0076425 A1
(43) Pub. Date: Mar. 19, 2009

(54) Equilateral Foot Bed and Systems Having Same

(75) Inventor: Richard Schwartz, Englewood, NJ (US)

Correspondence Address: MCDERMOTT WILL & EMERY LLP, 600 13TH STREET, N.W., WASHINGTON, DC, 20005-3096, US

(73) Assignee: AETREX WORLSWIDE, INC., Teaneck, NJ (US)

(21) Appl. No.: 12/085,143

(22) PCT Filed: Sep. 29, 2006
(86) PCT No.: PCT/US2006/038138

§ 371 Date: May 16, 2008

Related U.S. Application Data
(60) Provisional application No. 60/722,735, filed on Sep. 30, 2005.

(51) Int. Cl. A61F 5/052

(52) U.S. Cl. 602/23

(57) ABSTRACT

An equilateral foot bed and a system using the equilateral foot bed are provided for treating foot injuries and deformities, while allowing the patient to remain ambulatory. Embodiments include a foot bed having a first portion and a second portion, the second portion having a greater thickness than the first portion. The portions of differing thickness support, align and cushion portions of the foot. The foot bed is selectively usable in either a left foot-receiving or a right foot-receiving configuration.

### RELATED APPLICATIONS

This application claims priority to U.S. patent application Ser. No. 60/722,735, filed on Sep. 30, 2005, which Application is incorporated herein in its entirety by this reference.

### FIELD OF THE INVENTION

The present invention relates to devices for treating foot injuries and deformities. In particular, the present invention relates to a foot bed for use in treating foot injuries and deformities while allowing the patient to remain ambulatory, and treatment systems using the foot bed.

### BACKGROUND OF THE INVENTION

The human foot is a complex bio-mechanical system, which provides balance and mobility. When deformity, disease or injury imbalances the foot's structure, its supportive and shock-absorbing qualities are reduced and one suffers.

Foot deformity and disease afflicts a comparative few with long-term problems, however, many suffer short-term injuries involving feet. For example, patients having an ankle sprain or stress fractures of the lower leg or ankle remain ambulatory under contemporary treatment standards with the foot and ankle isolated by a “walker boot.” Typically, such boots are equilateral (that is, left-right interchangeable) and include a solid, one-piece rocker bottom portion having a flat foot-receiving portion and soft upper portions secured around the leg and foot by straps. In such boots, the patient's foot is well-constrained for bone and tendon healing, which may take weeks or months. Yet, for many patients, lack of proper foot support and cushioning in the constrained position makes walking in the “walker boot” uncomfortable.

Accordingly, what is needed is an economical, supportive and cushioning foot bed suitable for use with podiatric and orthopedic devices.

### SUMMARY OF THE INVENTION

Advantages and features of the present invention will be set forth in part in the description which follows and in part will become apparent to those having ordinary skill in the art upon examination of the following or may be learned from the practice of the invention. The advantages of the invention may be realized and obtained as particularly pointed out in the appended claims.

According to the present invention, the foregoing and other advantages are achieved in part by a foot bed extending longitudinally and laterally, and having a first portion and a second portion, the second portion having a greater thickness than the first portion, wherein the foot bed is selectively usable in either a left foot-receiving or a right foot-receiving configuration.

Another aspect of the present invention is a foot bed extending longitudinally and laterally and having non-uniform thickness about a medial surface, the thickness being to support and cushion portions of a foot, wherein the foot bed is substantially symmetrical about the medial surface.

A further aspect of the present invention is a system comprising a foot attachment and an equilateral foot bed positioned at a foot-receiving portion of the attachment, wherein the foot bed is for positioning and cushioning a foot.

Additional advantages of the present invention will become readily apparent to those skilled in this art from the following detailed description, wherein only an exemplary embodiment of the present invention is shown and described, simply by way of illustration of the best mode contemplated for carrying out the present invention. As will be realized, the present invention is capable of other and different embodiments, and its several details are capable of modifications in various obvious respects, all without departing from the invention. Accordingly, the drawings and description are to be regarded as illustrative in nature, and not as restrictive.

### BRIEF DESCRIPTION OF THE DRAWINGS

Aspects, advantages and novel features of the present invention will become apparent from the following description of the invention presented in conjunction with the accompanying drawings:

FIG. 1 is a perspective view of an embodiment of a walker boot and equilateral foot bed system in accord with the present invention;

FIG. 2A is a perspective view of an embodiment of an equilateral foot bed in accord with the present invention;

FIG. 2B is a top view of an embodiment of an equilateral foot bed in a left foot orientation;

FIG. 2C is a top view of an embodiment of an equilateral foot bed in a right foot orientation;

FIG. 2D is a cross-sectional view across a heel portion of an embodiment of an equilateral foot bed;

FIG. 2E is a cross-sectional view across an arch portion of an embodiment of an equilateral foot bed;

FIG. 2F is a cross-sectional view along a longitudinal axis of an embodiment of an equilateral foot bed;

FIG. 3A is a perspective view of an alternate embodiment of an equilateral foot bed in accord with the present invention;

FIG. 3B is a top view of an alternate embodiment of an equilateral foot bed in a left foot orientation;

FIG. 3C is a top view of an alternate embodiment of an equilateral foot bed in a right foot orientation;

FIG. 3D is a cross-sectional view across a heel portion of an alternate embodiment of an equilateral foot bed;

FIG. 3E is a cross-sectional view across an arch portion of an alternate embodiment of an equilateral foot bed;

FIG. 3F is a cross-sectional view along a longitudinal axis of an alternate embodiment of an equilateral foot bed

FIG. 4A is a perspective view of a second alternate embodiment of an equilateral foot bed in accord with the present invention;

FIG. 4B is a top view of a second alternate embodiment of an equilateral foot bed in a left foot orientation;

FIG. 4C is a top view of a second alternate embodiment of an equilateral foot bed in a right foot orientation;

FIG. 4D is a cross-sectional view across a heel portion of a second alternate embodiment of an equilateral foot bed;

FIG. 4E is a cross-sectional view across an arch portion of a second alternate embodiment of an equilateral foot bed;

FIG. 4F is a cross-sectional view along a longitudinal axis of a second alternate embodiment of an equilateral foot bed;

FIG. 4G is a cross-sectional view along a metatarsal portion of a second alternate embodiment of an equilateral foot bed

FIG. 5A is a perspective view of a third alternate embodiment of an equilateral foot bed in accord with the present invention;

FIG. 5B is a top view of a third alternate embodiment of an equilateral foot bed in a left foot orientation;

FIG. 5C is a top view of a third alternate embodiment of an equilateral foot bed in a right foot orientation;

FIG. 5D is a cross-sectional view across a heel portion of a third alternate embodiment of an equilateral foot bed;

FIG. 5E is a cross-sectional view across an arch portion of a third alternate embodiment of an equilateral foot bed;

FIG. 5F is a cross-sectional view along a longitudinal axis of a third alternate embodiment of an equilateral foot bed

FIG. 6A is a perspective view of an embodiment of a fourth alternate equilateral foot bed in accord with the present invention;

FIG. 6B is a top view of an embodiment of a fourth alternate equilateral foot bed in a left foot orientation;

FIG. 6C is a top view of an embodiment of a fourth alternate equilateral foot bed in a right foot orientation;

FIG. 6D is a cross-sectional view across a heel portion of a fourth alternate embodiment of an equilateral foot bed;

FIG. 6E is a cross-sectional view across an arch portion of a fourth alternate embodiment of an equilateral foot bed;

FIG. 6F is a cross-sectional view along a longitudinal axis of a fourth alternate embodiment of an equilateral foot bed FIG. 7A is a perspective view of a fifth alternate embodiment of an equilateral foot bed in accord with the present invention;

FIG. 7B is a top view of a fifth alternate embodiment of an equilateral foot bed in a left foot orientation;

FIG. 7C is a top view of a fifth alternate embodiment of an equilateral foot bed in a right foot orientation;

FIG. 7D is a cross-sectional view across a heel portion of a fifth alternate embodiment of an equilateral foot bed;

FIG. 7E is a cross-sectional view across an arch portion of a fifth alternate embodiment of an equilateral foot bed; and

FIG. 7F is a cross-sectional view along a longitudinal axis of a fifth alternate embodiment of an equilateral foot bed

### DESCRIPTION OF THE INVENTION

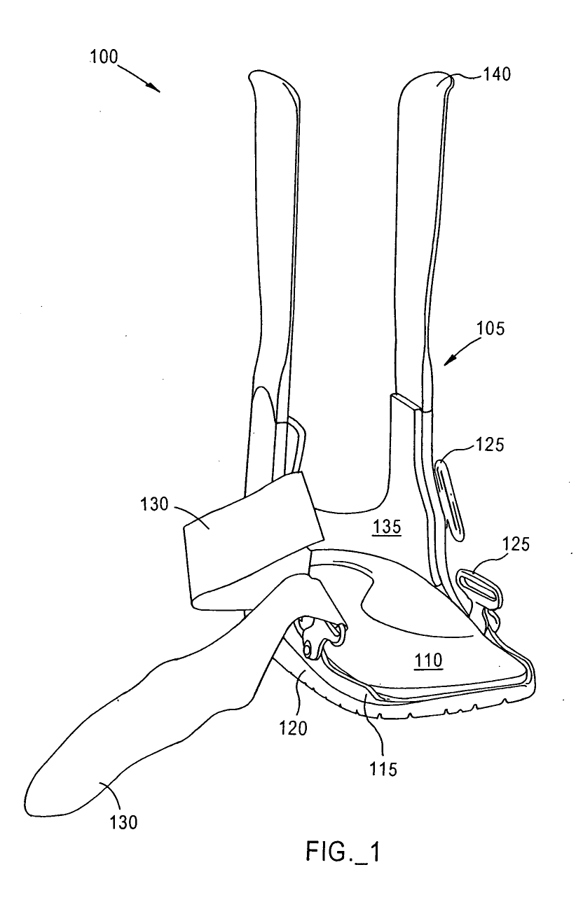

FIG. 1 is a perspective view of an embodiment of a walker boot and foot bed system in accord with the present invention. In this embodiment, which like all of the embodiments described below is a teaching example of broader principles of the invention, podiatric/orthopedic system 100 includes walker boot 105 and equilateral foot bed 110. The walker boot and foot bed are optionally secured together by adhesive tape or the like. Instead of a walker boot, alternate embodiments of the inventive system include cast walkers, or ankle high walker boots, or post-operative shoes, or cast boots, or night splints, or post-operative splints. Generically, all attachments to an immobilized foot are in accord with the invention.

In FIG. 1, walker boot 105 is an otherwise customary device including foot-receiving portion 115, sole 120, buckle 125, strap 130, ankle portion 135 and tibia portion 140. Typically, the foot-receiving portion is flat and equilateral. That is, the foot-receiving portion and thus the walker boot are equally appropriate for use on either a right or a left foot.

To wear this example of walker boot 105, a patient secures the walker boot to their lower leg and foot by straps 130 and buckles 125 in addition to other strapping around the lower leg (not shown for clarity). Soft material (not shown for clarity) above the foot and around the leg is also commonly used underneath the straps or other securing means.

As is well-known in medical practice, walker boot 105 constrains a foot and ankle for healing while allowing a patient to remain ambulatory during recovery. Often, recovery periods are weeks or months long. Experience shows that without equilateral foot bed 110, that is with a bare foot-receiving portion 115, the constraining effects of the walker boot malposition the foot with respect to performing it's natural shock absorbing and support functions. Such malpositioning often leads to patient discomfort when walking. As a result, the promise of the walker boot in enabling ambulatory patient care is not fully realized because patients walk less than they might due to discomfort.

To mitigate foot malpositioning and its attendant discomfort, the structure of equilateral foot bed 110 supports and cushions a foot, performing a proper biomechanical alignment of the entire leg. As a result, a patient is more comfortable. Such a comfort advantage is a potent product-differentiating feature. Moreover, with less discomfort, a patient may walk more during healing, thereby more fully realizing the promise and benefits of ambulatory recovery. Additionally, since the inventive foot bed is equilateral, its cost is lower than prior-art foot beds that are configured for only the left or the right foot.

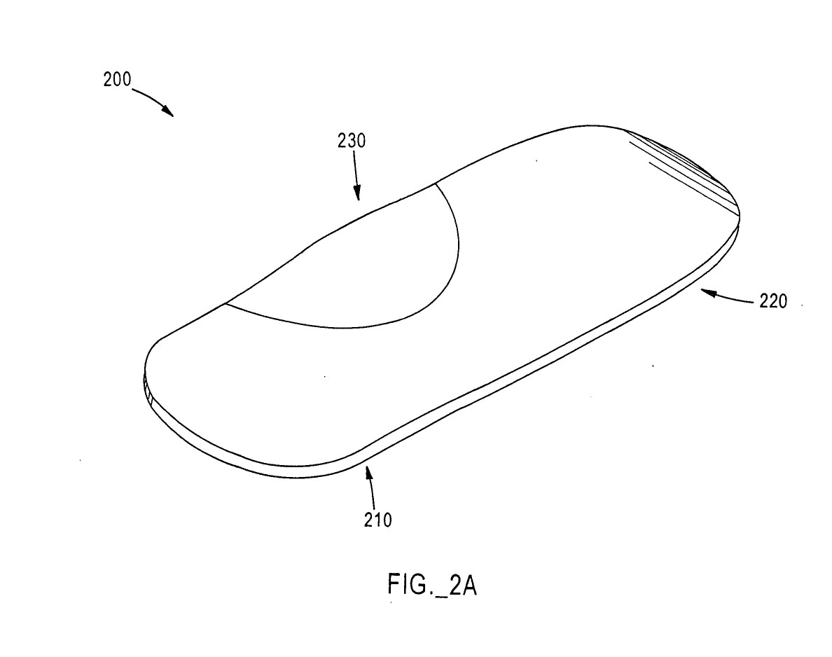

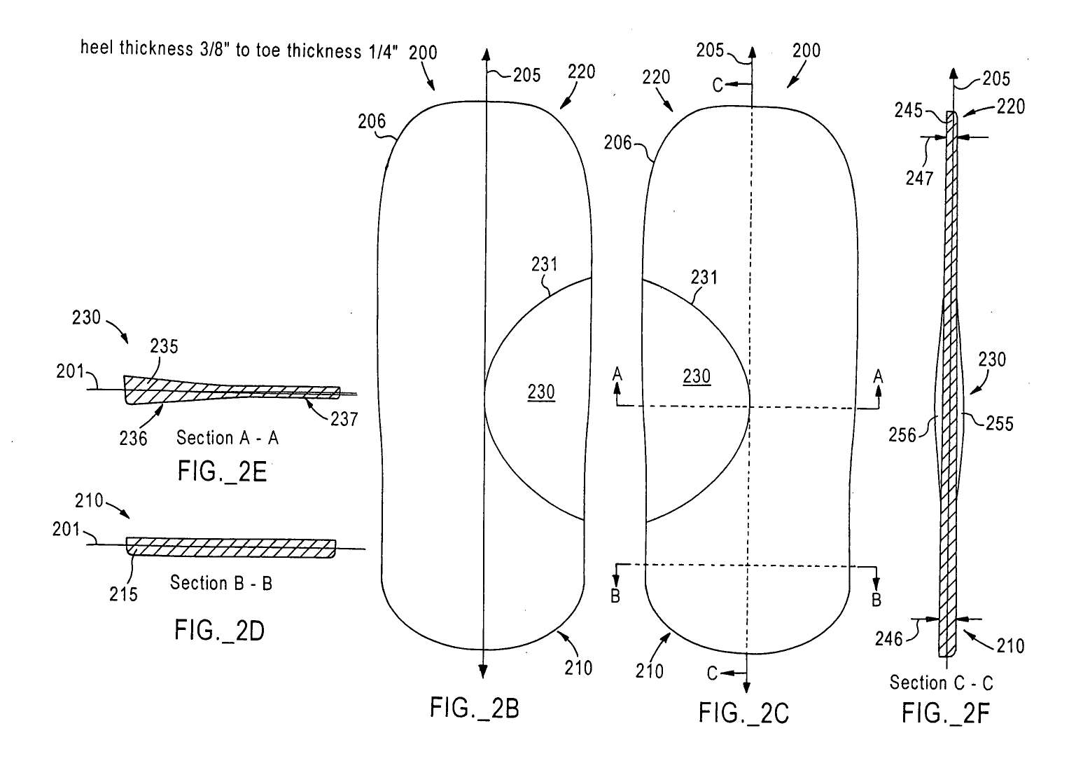

FIG. 2A is a perspective view of an embodiment of an equilateral foot bed in accord with the present invention. In this first of several teaching examples, similar figures and elements are ascribed similar lettering and numbers, respectively. Here, equilateral foot bed 200 extends longitudinally and laterally in an oblong manner, as a foot, and includes heel portion 210, toe portion 220 and arch portion 230. Alternate embodiments do not extend sufficiently to provide a bed for an entire foot, for example by omitting part or all or toe portion 220. Different embodiments of the foot bed also have differing lengths and widths to accommodate a range of foot sizes in the population and discrete size categories (S, M, L, XL, for example) of existing walker boots and other devices.

Typically, equilateral foot bed 200 is made of one or more cushioning and supportive materials, such as elastometric materials; for example, synthetic rubbers. Other embodiments are made, in whole or in part, of gel, or foams, or leather or plastics and the like. In some embodiments, gels are selectively included in heel portion 210 or, likewise, forward toward toe portion 220 near a ball of the foot region or a metatarsal region. Preferably, but not necessarily, the foot bed is manufactured by molding processes.

FIG. 2B and FIG. 2C are top views of equilateral foot bed 200 in a left foot orientation and a right foot orientation, respectively. To reorient between left and right foot orientations, one rotates the foot bed around longitudinal axis 205.

FIG. 2B and FIG. 2C both show foot bed perimeter 206, which has substantially straight side portions and arcuate heel and toe portions. Such a geometry is appropriate for many commercially available walker boots. In alternate embodiments, however, the foot bed perimeter is differently shaped to accommodate other components of a podiatric or orthopedic system. Many variations will be apparent to a skilled person.

FIG. 2B and FIG. 2C also show arch portion 230 bounded to the interior of the foot bed 200 by arch perimeter 231. When engaged with a foot, the arch portion substantially underlies an arch of the foot. In this example, the arch perimeter is a substantially symmetrical arc. Other embodiments however, have different geometries. Again, many variations will be apparent to a skilled person.

FIG. 2D is a cross-sectional view across heel portion 210 of equilateral foot bed 200. See FIG. 2C for cross section B-B, to which FIG. 2D corresponds. In this embodiment, heel portion cross section 215 has a substantially constant area across the lateral extent of the cross section. For the embodiment shown in FIG. 2D, the thickness of the heel portion is about ⅜ inch. Alternate embodiments have thinner heel portion thickness, such as 1/16 to 5/16 of an inch. Other embodiments have a thicker heel portion, such as, for example 7/16 inch to 1 inch. Embodiments suitable for patients with a leg length disparity have a heel portion greater than 1 inch.

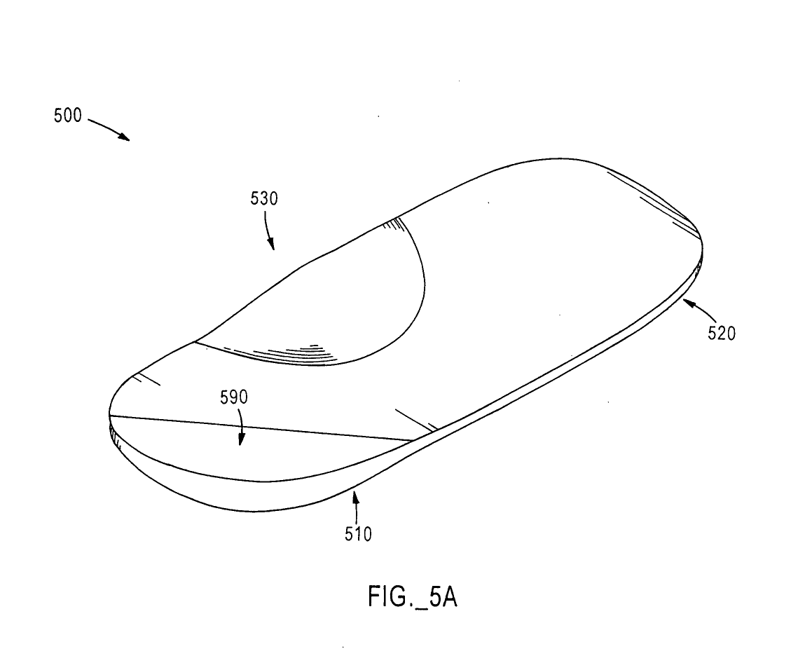

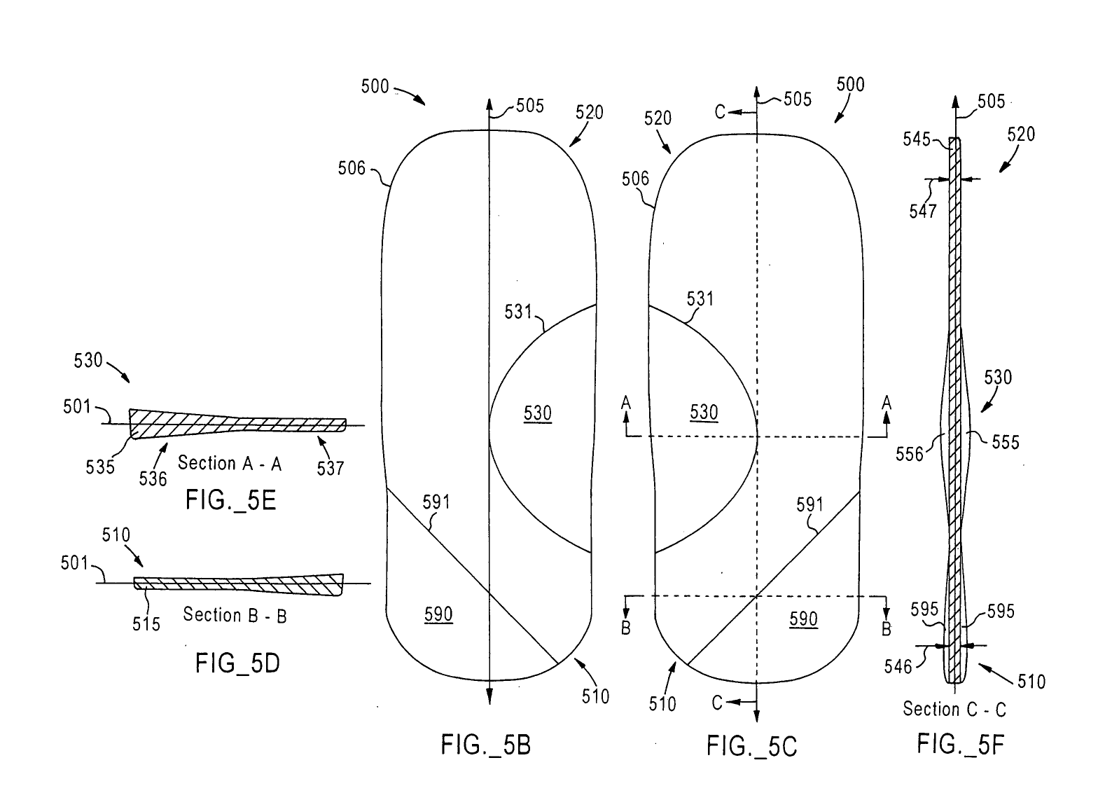

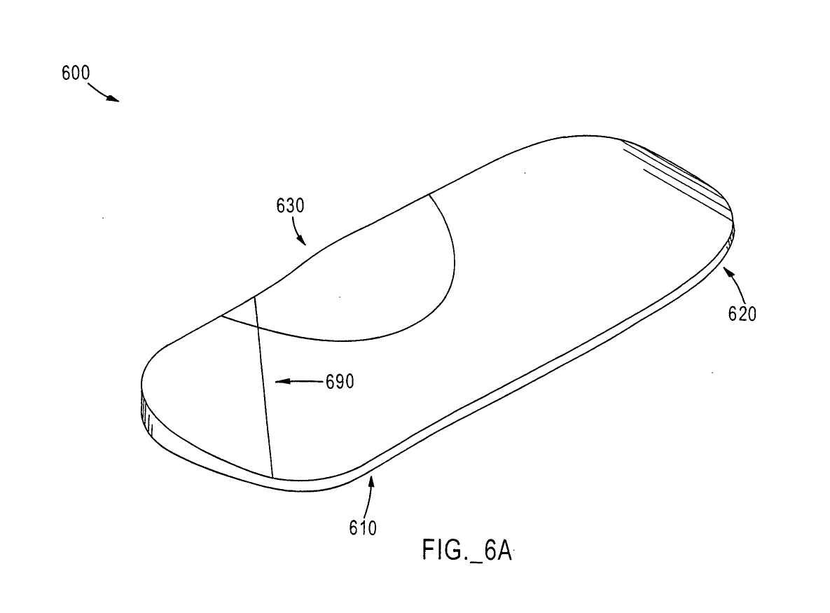

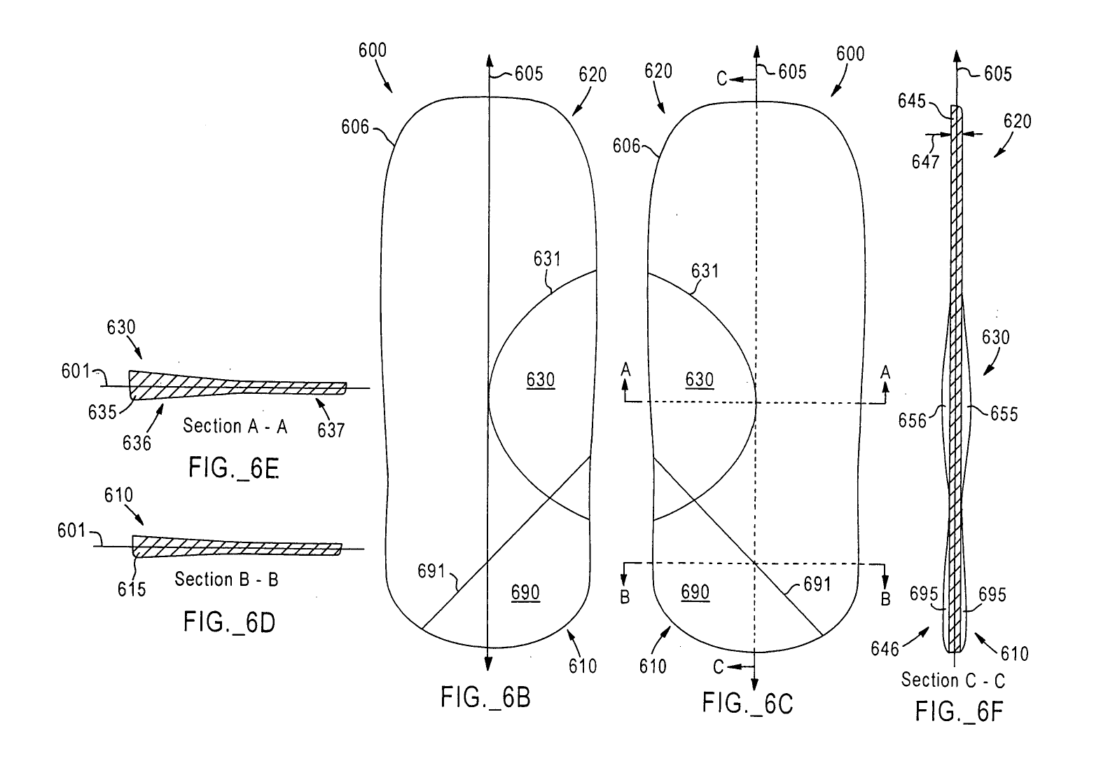

The embodiment in FIG. 2D has heel portion cross section 215 with a substantially constant area across the lateral extent of the cross section. In other embodiments, however, cross sections across the heel portion have a change in area, for example due to a heel pad or a depression. In different embodiments, the cross sectional area change is symmetrical about longitudinal axis 205 (see FIG. 2C) or unsymmetrical (see, for example, FIG. 5D and FIG. 6D).

FIG. 2E is a cross-sectional view across arch portion 230 of equilateral foot bed 200. See FIG. 2C for cross section A-A, to which FIG. 2E corresponds. In FIG. 2E, arch portion cross section 235 has a cross-sectional area change moving laterally from outside portion 237 to inside portion 236, providing a supporting structure for the arch of the foot. In FIG. 2E, the change in cross sectional area is substantially linear. Other embodiments vary non-linearly. Typical arch thicknesses are from zero to one inch, preferably ½ inch. Embodiments suitable for patients with a foot deformity can have an arch thickness greater than an inch, as needed. Many variations are possible in accord with the invention.

FIG. 2F is a cross-sectional view along longitudinal axis 205 of equilateral foot bed 200. See FIG. 2C for cross-section C-C, to which FIG. 2F corresponds. In FIG. 2F, cross sectional area 245 changes from heel portion 210 towards toe portion 220 as foot bed thickness varies from about ⅜ inch at heel thickness 246 to about ¼ inch at toe thickness 247. Such a tapered foot bed is advantageous to improve gait mechanics. While typical tapering has thickness decreasing from heel towards toe, other embodiments have thickness increasing from heel towards toe. Still other embodiments, as described below, have equal heel and toe thicknesses. For a specific application, the tapering is determined by factors such as heel height of a walker boot or other device used in combination with the foot bed, patient foot shape and prescriptive treatment objectives of a medical practitioner.

FIG. 2B-FIG. 2F illustrate a substantial symmetry of foot bed 200 about medial surface 201 (see FIG. 2D and FIG. 2E). That is, foot bed 200 has two portions on either side of the medial surface which are largely, if not exactly, the same. As a result of such symmetry, one foot bed is equally appropriate for use with either a left or a right foot. That is, the foot bed is equilateral. To reorient from left foot to right foot orientation, one need only rotate the foot bed about longitudinal axis 205 (see FIG. 2C).

Depending on the foot bed manufacturing process, otherwise insubstantial deviations from symmetry are advantageous. For example, foot beds made by a molding process can be easier to remove from a mold if the two portions of the foot bed on either side of the medial surface are not exactly the same. Nevertheless, a range of embodiments of the equilateral foot bed have the two portions on either side of the medial surface the same, within engineering tolerances.

As described, substantial symmetry about a medial surface, for example without limitation, a plane, enables an equilateral foot bed. When foot bed 200 is secured to foot-receiving portion 115 in exemplary podiatric system 100 (see FIG. 1), the foot bed deforms to comply with the foot-receiving portion, and arch support portions 255 and 256 on both sides of medial surface 201 contribute to the support and cushioning functionality of the foot bed (see FIG. 2F). In the instance of arch portion 230 in FIG. 2E, when secured to a flat foot bed in a podiatric system, the arch portion will bend upward to comply with the flat foot-receiving portion, and foot bed material on either side of the medial surface (now curved) contributes to supporting the arch of the foot.

Such an equilateral foot bed is advantageous in several respects. First, because many existing podiatric devices, such as walker boots, are equilateral, an equilateral foot bed is universal, easily fitting to existing devices of any brand as well as newly manufactured units and future designs. Moreover, the equilateral foot bed is universal to the range of patient feet. All kinds of sizes and deformities can be fit. Second, in regard to manufacturing as well as inventory and supply chain costs, an equilateral foot bed is less expensive than right/left specific products. Third, by mitigating foot discomfort, the equilateral foot bed improves patient compliance with a medical practitioner's instructions as to walking. Accordingly, by providing significantly increased patient comfort at a small price differential, podiatric systems with an equilateral foot bed, such as the example shown in FIG. 1, are a compelling product offering to patients, medical practitioners and suppliers alike.

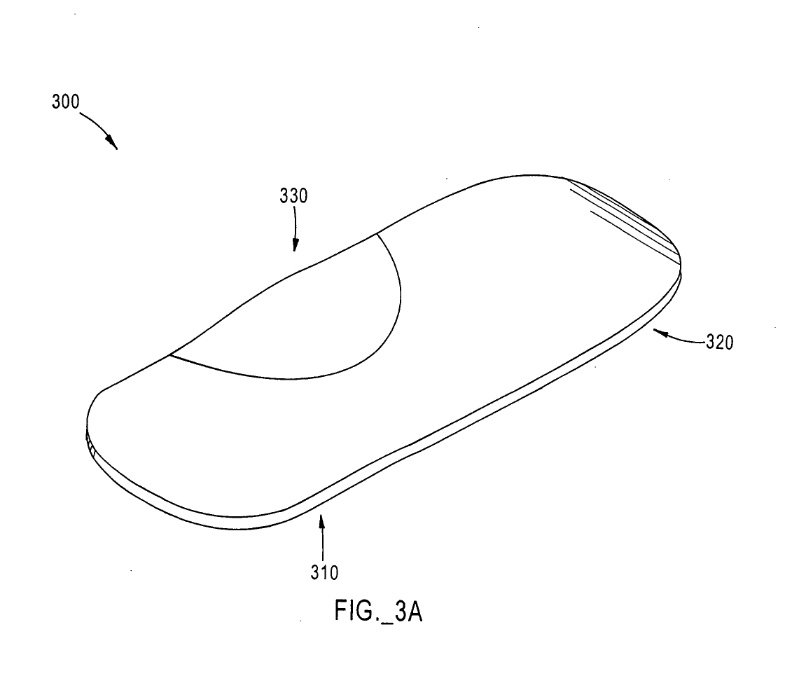

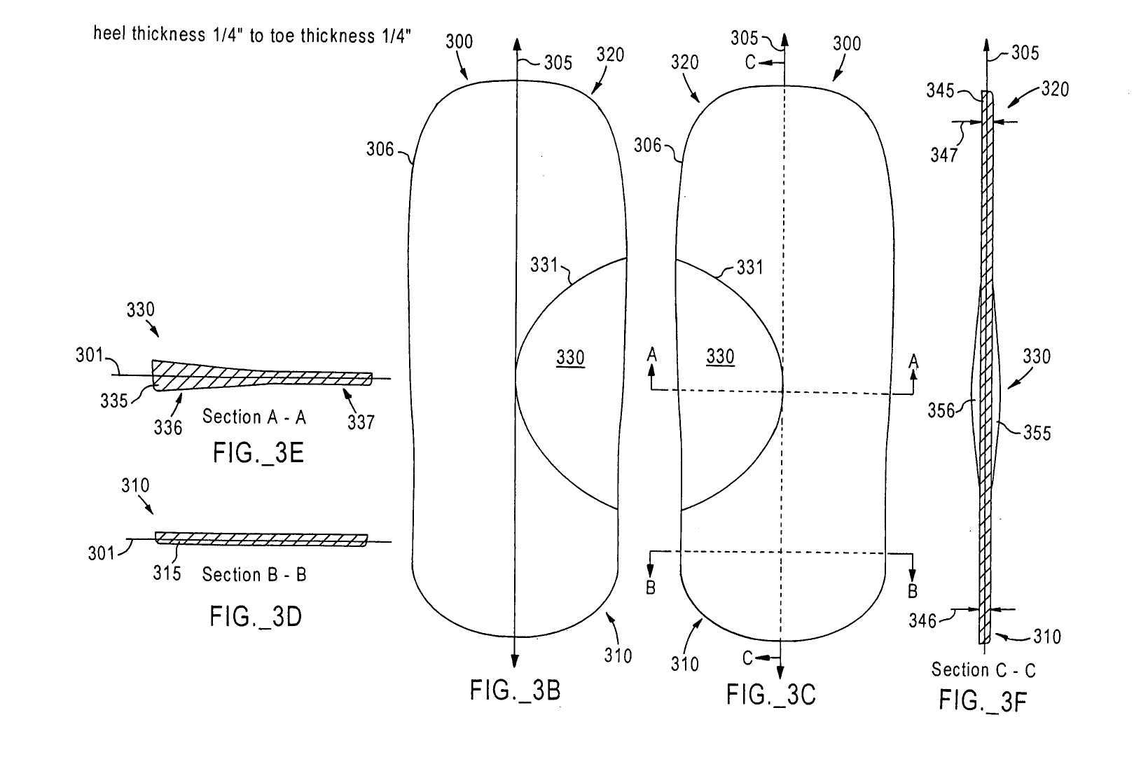

FIG. 3A-FIG. 3F illustrate an alternate embodiment of an equilateral foot bed in accord with the present invention. This alternate embodiment is similar to the embodiment shown in FIG. 2A-FIG. 2F, except that heel and toe thicknesses 346 and 347, respectively, are the same (see FIG. 3F). Accordingly, like figures share the same letter (for example, FIG. 2B corresponds to FIG. 3B) and like elements correspond except for a leading digit signifying the figure number (for example, 247 in FIG. 2F corresponds to 347 in FIG. 3F). The descriptions of the figures also correspond, except that there is no heel-to-toe taper of cross sectional area 345. Rather, heel thickness 346 is about ¼ inch and toe thickness is about ¼ inch (see FIG. 3F). Other embodiments have equal dimensions across a broad range from about 1/16 inch to greater than 1 inch for patients with leg length disparity.

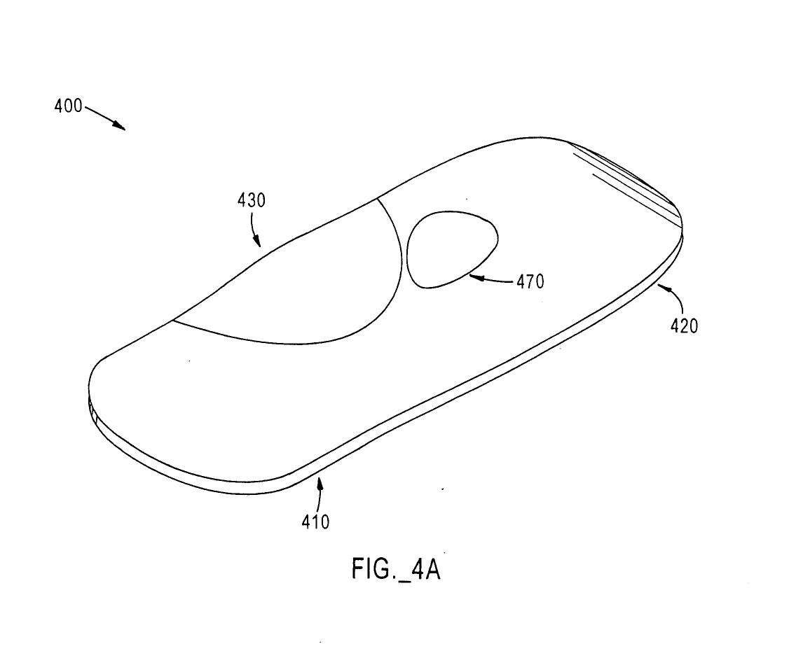

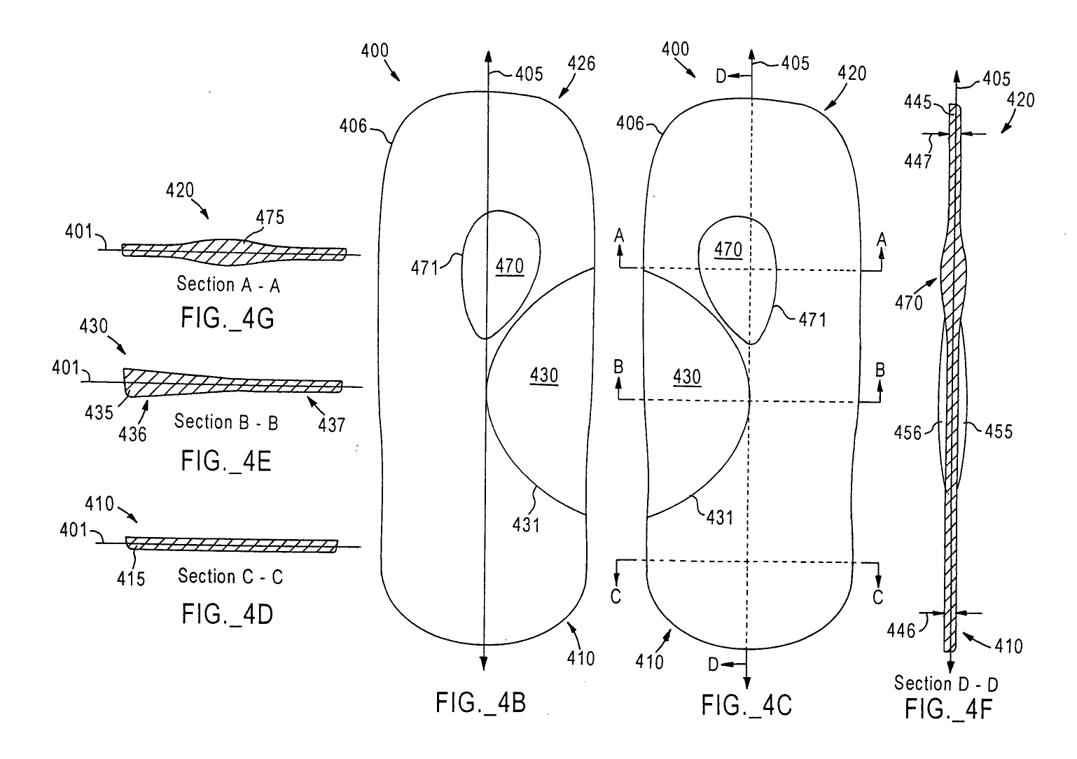

FIG. 4A is a perspective view of a second alternate embodiment of an equilateral foot bed in accord with the present invention. As in the previously described embodiments, equilateral foot bed 400 includes heel portion 410, toe portion 420 and arch portion 430. The embodiment in FIG. 4A further includes metatarsal portion 470 located in proximity to the arch portion, the degree of proximity determined by well-known human anatomy and desired foot sizing ranges. As arch portion 430 provides cushioned support to the arch of the foot, so does the metatarsal portion provide cushioned support to the metatarsals,

As with the previously described embodiments, FIG. 4B and FIG. 4C are top views showing equilateral foot bed 400 in left foot and right foot orientations, respectively. In this embodiment, metatarsal perimeter 471 bounds metatarsal portions 470 in an arcuate shape, much like an egg silhouette, with a broader portion forward toward toe portion 420 and a narrower portion pointing toward heel portion 410, the pointing in a manner skewed toward an outward side of the foot bed. Other embodiments have differently shaped metatarsal portions consistent in shape and placement on the foot bed with well-known human anatomy and it's variations. For example, other embodiments have metatarsal portions in oval or oblong or teardrop shapes, all of which extend laterally to an extent sufficient to cushion and support from one to five metatarsals. Many shapes and positions are in accord with the invention.

FIG. 4D and FIG. 4E are cross-sectional views across a heel portion and an arch portion of foot bed 400, respectively. The description of these figures is similar to the description of FIG. 2D and FIG. 2E, above.

FIG. 4F is a cross-sectional view along longitudinal axis 405 of equilateral foot bed 400. See the description of FIG. 2F for similar aspects. Referring to FIG. 4F, cross-sectional area 445 bulges across the cross-section of metatarsal portion 470. As with arch support portions 455 and 456, the extent of the bulge in the metatarsal portion's cross section differs in different embodiments according to a patient's foot anatomy and cushioning and support needs in the metatarsal area. In FIG. 4F, the metatarsal portion's bulge is about that of arch support portions 455 and 456, which typically is about ¼ inch on either side of medial surface 401. In other embodiments, the metatarsal portion's bulge is about 1/16 inch or less, or up to about ½ inch. For severe foot deformities, the metatarsal portion's bulge can exceed ½ inch.

FIG. 4G is a cross-sectional view along metatarsal portion 470 of equilateral foot bed 400. See FIG. 4C for section A-A, to which the cross-sectional view corresponds. As was the case in the longitudinal cross section of FIG. 4F, the lateral cross-section of FIG. 4G illustrates the bulging increase in cross-sectional area 475 due to metatarsal portion 470.

As in the previous embodiments, the structure of metatarsal portion 470 has substantial symmetry about medial surface 401. As described above with respect to the arch portion, when foot bed 400 is secured to foot-receiving portion 115 in exemplary podiatric system 100 (see FIG. 1), the foot bed deforms to comply with the foot-receiving portion, and portions of metatarsal portion 470 on both sides of medial surface 401 contribute to the support and cushioning functionality of the foot bed (see FIG. 4F and FIG. 4G), in particular supporting and cushioning the metatarsals.

The embodiment shown in FIG. 4A-FIG. 4F includes foot bed portions that provide additional material thickness for cushioning and support. In further alternative embodiments, portions of the foot bed have reduced or no thickness (a hole) to accommodate foot deformities such as “rocker bottom” foot. As with additional material thickness, many variation of reduced thickness are in accord with the invention.

FIG. 5A-FIG. 5F and FIG. 6A-FIG. 6F illustrate two alternative embodiments of an equilateral foot bed having heel wedge portions, 590 and 690, respectively. In both instances, the heel wedge portions provide an increase in material thickness in select areas of the foot bed. As in the previous embodiments, the structures of heel wedge portions 590 and 690 have substantial symmetry about medial surfaces 501 and 601, respectively. See FIG. 5D and FIG. 6D.

While the embodiments in FIG. 5A-FIG. 5F and FIG. 6A-FIG. 6F show linearly ramping heel wedge portions, other embodiments have non-linear variation. Further, wedge perimeter 591 and 691 can be non-linear. Still further ranges of embodiments have heel pads instead of wedges, which pads are made of gel or the like. Different embodiments have pads which are symmetric or asymmetric with respect to a longitudinal axes corresponding to longitudinal axis 505 and the pads extend across the entire heel portion or a fraction of the heel portion. Moreover, such heel pads are included in embodiments having either a heel portion thickness greater than a toe portion thickness, or a toe portion thickness greater than a heel portion thickness.

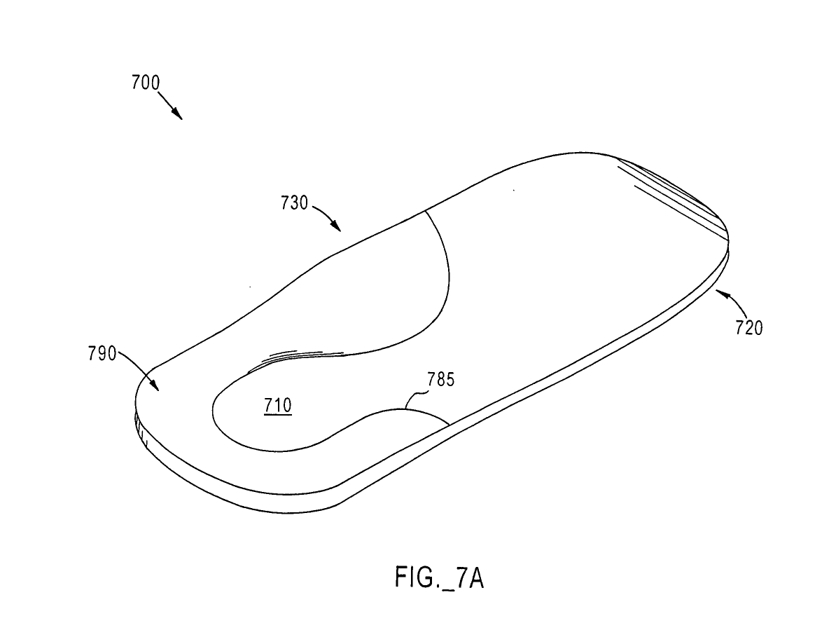

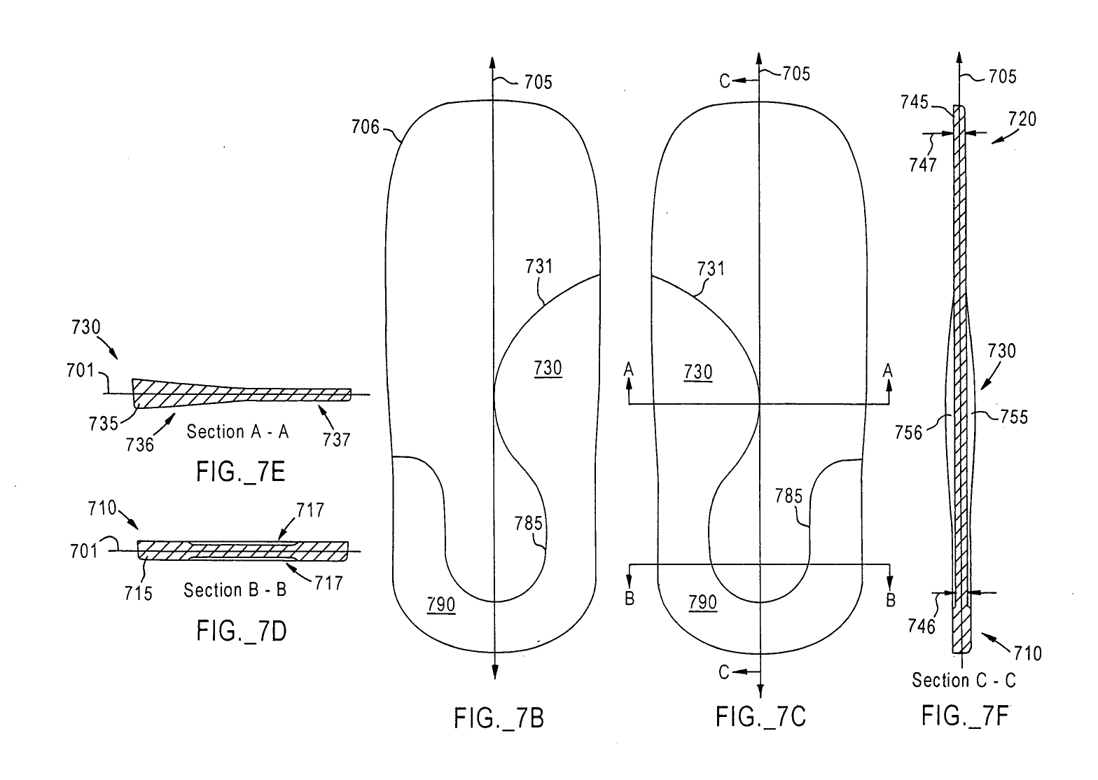

FIG. 7A-FIG. 7F illustrate yet another alternative embodiment of an equilateral foot bed in accord with the present invention. As compared to the embodiment in FIG. 2A-FIG. 2F, the embodiment in FIG. 7A-FIG. 7F includes raised portion 790, which is elevated above heel portion 710. Optionally, raised portion 790 is above heel portion 710 by greater than about 1/10 of the thickness of heel portion 710. In most embodiments, raised portion 790 is no more than ¼ inch above heel portion 710. Raised portion perimeter 785 has an arcuate, horseshoe-like shape largely defining a heel position before merging with arch portion 730 and arch perimeter 731. In both its elevation over heel portion 710 and the shape of the perimeter, raised portion 785 acts as a positioning guide to center a patient's heel over foot bed 700 as the heel begins to engage foot bed 700. While the embodiment shown has raised portion perimeter 785 with an arcuate, horseshoe-like shape, different embodiments have differing raised portion geometries. Many variations are in accord with the invention.

As in the previous embodiments, the structure of the embodiment in FIG. 7A-7F has substantial symmetry about medial surface 701. See FIG. 7D-FIG. 7E.

The present invention can be practiced by employing conventional materials, methodology and equipment. Accordingly, the details of such materials, equipment and methodology are not set forth herein in detail. In the previous descriptions, numerous specific details are set forth, such as specific materials, structures, chemicals, processes, etc., in order to provide a thorough understanding of the present invention. However, it should be recognized that the present invention can be practiced without resorting to the details specifically set forth. In other instances, well known processing structures have not been described in detail, in order not to unnecessarily obscure the present invention.

Only exemplary embodiments of the present invention and but a few examples of its versatility are shown and described in the present disclosure. It is to be understood that the present invention is capable of use in various other combinations and environments and is capable of changes or modifications within the scope of the inventive concept as expressed herein.

What is claimed is:

1. A foot bed extending longitudinally and laterally, and having a first portion and a second portion, the second portion having a greater thickness than the first portion, wherein the foot bed is selectively usable in either a left foot-receiving or a right foot-receiving configuration.

2. The foot bed of claim 1, wherein a rotation about a longitudinal axis of the foot bed selects between the left foot-receiving and the right foot-receiving configuration.

3. The foot bed of claim 1, wherein the second portion includes a toe portion.

4. The foot bed of claim 1, wherein the foot bed includes at least one of elastomer, gel, foam, leather and plastic.

5. The foot bed of claim 1, comprising a foot bed perimeter having straight side portions and an arcuate heel portion.

6. The foot bed of claim 1, wherein the second portion includes an arch-supporting portion.

7. The foot bed of claim 1, wherein the second portion includes a metatarsal-supporting portion.

8. The foot bed of claim 1, wherein the second portion includes a heel portion.

9. The foot bed of claim 8, wherein a lateral cross section across the heel portion has a varying cross-sectional area.

10. The foot bed of claim 1, wherein the second portion comprises a wedge-shaped heel portion.

11. The foot bed of claim 10, wherein the wedge-shaped heel portion has a non-linear wedge perimeter.

12. The foot bed of claim 1, wherein a longitudinal cross section has a tapered cross sectional area.

13. The foot bed of claim 1, wherein the foot bed is for receiving a deformed foot.

14. The foot bed of claim 13, wherein the foot bed is perforated to receive a deformed foot.

15. The foot bed of claim 1, wherein the second portion includes a guide portion to receive and position a foot over the foot bed.

16. A system, comprising a foot attachment and the foot bed of claim 1 positioned at a foot-receiving portion of the attachment.

17. A foot bed extending longitudinally and laterally and having non-uniform thickness about a medial surface, the thickness being to support and cushion portions of a foot, wherein the foot bed is substantially symmetrical about the medial surface.

18. The foot bed of claim 17, wherein the supported and cushioned portions of the foot include a heel portion.

19. The foot bed of claim 17, wherein the supported and cushioned portions of the foot include a toe portion.

20. The foot bed of claim 17, wherein the supported and cushioned portions of the foot include an arch portion.

21. The foot bed of claim 17, wherein the supported and cushioned portions of the foot include a metatarsal portion.

22. The foot bed of claim 18, wherein a lateral cross section across the heel portion has a varying cross-sectional area.

23. The foot bed of claim 18, wherein the heel portion is wedge-shaped.

24. The foot bed of claim 23, wherein the wedge-shaped heel portion has a non-linear wedge perimeter.

25. The foot bed of claim 17, wherein the foot bed comprises at least one of elastomer, gel, foam, leather and plastic.

26. The foot bed of claim 17, comprising a foot bed perimeter having substantially straight side portions and an arcuate heel portion.

27. The foot bed of claim 17, wherein a longitudinal cross section has a tapered cross-sectional area.

28. The foot bed of claim 17, wherein the foot bed is for receiving a deformed foot.

29. The foot bed of claim 28, wherein the foot bed is perforated to receive a deformed foot.

30. The foot bed of claim 17, comprising a guide portion to receive and position a foot over the foot bed.

31. A system, comprising a foot attachment and the foot bed of claim 17 positioned at a foot-receiving portion of the attachment.

32. A system, comprising a foot attachment and an equilateral foot bed positioned at a foot-receiving portion of the attachment, wherein the foot bed is for positioning and cushioning a foot.

33. The system of claim 32, wherein the foot attachment comprises one of a walker boot, a cast walker, a post-operative shoe, a cast boot, and a splint.
